(19) United States
(12) Patent Application Publication
(10) Pub. No.: US 2024/0382538 A1
(43) Pub. Date: Nov. 21, 2024

(54) AGENT FOR PREVENTION AND/OR TREATMENT OF PORPHYROMONAS GINGIVALIS INFECTION

(71) Applicant: NUTRI CO., LTD., Yokkaichi-shi, Mie (JP)

(72) Inventor: Susumu KAWAGUCHI, Yokkaichishi, Mie (JP)

(73) Assignee: NUTRI CO., LTD., Yokkaichi-shi, Mie (JP)

(21) Appl. No.: 18/685,863

(22) PCT Filed: Aug. 18, 2022
(86) PCT No.: PCT/JP2022/031241

§ 371 Date: Feb. 22, 2024

(30) Foreign Application Priority Data
Aug. 27, 2021 (JP) 2021-139391

(51) Int. Cl. A61K 35/744; A61P 31/04

(52) U.S. Cl. CPC A61K 35/744; A61P 31/04

(57) ABSTRACT

Provided is a prophylactic and/or therapeutic agent for Porphyromonas gingivalis infection. A prophylactic and/or therapeutic agent for Porphyromonas gingivalis infection, comprising killed Enterococcus faecalis. A prophylactic and/or therapeutic agent for a disease attributable to Porphyromonas gingivalis, comprising killed Enterococcus faecalis. A medicine or food for prevention and/or treatment of Porphyromonas gingivalis infection, comprising killed Enterococcus faecalis. A medicine or food for prevention and/or treatment of a disease attributable to Porphyromonas gingivalis, comprising killed Enterococcus faecalis. A growth inhibitor for Porphyromonas gingivalis, comprising killed Enterococcus faecalis. An inhibitor for gingipain activity, comprising killed Enterococcus faecalis.

### TECHNICAL FIELD

The present invention relates to a prophylactic and/or therapeutic agent for Porphyromonas gingivalis infection.

### BACKGROUND ART

Porphyromonas gingivalis (hereinafter, referred to as “P. gingivalis”), which is a gram negative, obligate anaerobe, is a representative pathogenic bacterium believed to be most largely involved in the onset and the progression of periodontitis. Periodontitis is a chronic inflammatory disease that is observed in the gingival tissue of approximately two-thirds of persons over 30 years old in Japan¹⁾. Being isolated from the flora in subgingival lesions, saliva, and the surface of tongue, tonsils, etc. of adult periodontitis patients. P. gingivalis induces inflammation to cause breakdown of the immune system²⁾, so it is known to be strongly associated with not only oral diseases but also various systemic diseases^{3,4)}. Specifically, P. gingivalis has been pointed out as a risk factor for severe systemic diseases including cardiovascular diseases (endocarditis, coronary heart disease, etc.), pneumonia, preterm delivery, and delivery of low weight babies. It is also said that P. gingivalis is detected in 45% of atherosclerotic lesions. While association between P. gingivalis and systemic diseases has heretofore been pointed out, pathogenic factors such as gingipain that is a powerful protease secreted by P. gingivalis on its cell surfaces or extracellularly have recently been found in the brain of Alzheimer's disease patients and in Alzheimer's disease model animals⁵⁻⁹⁾ and, therefore, association between Alzheimer's disease and chronic periodontitis that is believed to be caused by P. gingivalis infection is also attracting attention⁶⁾.

One of the main features of P. gingivalis is that it does not have sugar fermentation ability but produces and secretes the protease gingipain on its cell surface and outside the cell, whereby it depends on proteins and peptides of the external world for nutrition and energy source ¹⁰⁾. This gingipain eliminates the adhesive property of gingival fibroblasts and vascular endothelial cells to thereby destroy the periodontal tissue both structurally and functionally. Furthermore, gingipain has a strong pathogenicity that induces various pathological conditions as exemplified by causing degradation of host proteins, blood coagulation, increased vascular permeability, leukocyte dysfunction and host cell death^{11), 12)}. Depending on the specificity of peptide cleavage site, gingipain has two enzyme groups: Arg-gingipain (Rgp) which cleaves the C-terminus of arginine residue, and Lys-gingipain (Kgp) which cleaves the C-terminus of lysine residue ¹³⁾. Since these enzyme groups are not inactivated but can maintain and exert their activity in the living body, they cause damage to host cells but at the same time, they are essential for the survival and proliferation of the bacterium itself. It is known that the growth of P. gingivalis can be inhibited by using a specific inhibitor of gingipain¹⁴⁾ or by knocking out the gingipain gene, so gingipain is indeed essential for the growth of this bacterium¹³⁾.

It has already been revealed in tests using infected model animals that oral administration of Enterococcus faecalis (Heat-killed lactic acid bacterium Enterococcus faecalis; hereinafter, referred to as “HkEf”) has phylactic effect¹⁵⁾¹⁶) on infections with methicillin-resistant Staphylococcus aureus (MRSA), multidrug-resistant Pseudomonas aeruginosa (MDRP), Streptococcus pneumoniae (S. pneumoniae), and Clostridioides difficile (C. difficile), all of which are pathogens causing hospital infections. Further, a direct growth-inhibiting effect17) on C. difficile has been confirmed in an in vitro test using mixed culture.

### PRIOR ART LITERATURE

### Non-Patent Documents

Non-Patent Document No. 1:2016 Survey of Dental Diseases (Ministry of Health, Labor and Welfare)

Non-Patent Document No. 2: Maekawa T, Krauss J L, Abe T et al., Porphyromonas gingivalis manipulates complement and TLR signaling to uncouple bacterial clearance from inflammation and promote dysbiosis. Cell Host Microbe. 15:768-778, 2014.

Non-Patent Document No. 3: Aoyama N, Suda T, Ikeda Y et al., Survey on Systemic Conditions of New Patients Who Visited Periodontitis Outpatient Department of the Tokyo Medical and Dental University Hospital, J. Stomatol. Soc. Jpn., 84, 37-44, 2017.

Non-Patent Document No. 4: Kimura Y, Shimabara S, Hashimoto N et al., Relationship between Oral Diseases and the Whole Body-Importance of Oral Care, Bulletin of Aino Gakuinn, 27:9-17, 2013.

Non-Patent Document No. 5: Ilievski V, Zuchowska P K, Green S J et al. Chronic oral application of a periodontal pathogen results in brain inflammation, neurodegeneration and amyloid beta production in wild type mice. PLOS One. 13, e0204941, 2018.

Non-Patent Document No. 6: Dominy S S, Lynch C, Ermini F et al., Porphyromonas gingivalis in Alzheimer's disease brains: Evidence for disease causation and treatment with small-molecule inhibitors. Sci Adv. 5: eaau3333, 2019.

Non-Patent Document No. 7: Poole S, Singhrao S K, Kesavalu L et al., Determining the Presence of Periodontopathic Virulence Factors in Short-Term Postmortem Alzheimer's Disease Brain Tissue. J Alzheimers Dis. 36:665-677, 2013.

Non-Patent Document No. 8: Poole S, Singhrao S K, Chukkapalli S et al., Active Invasion of Porphyromonas gingivalis and Infection-Induced Complement Activation in ApoE-/-Mice Brains. J Alzheimers Dis. 43, 67-80, 2015.

Non-Patent Document No. 9: Nie R, Wu Z, Ni J et al., Porphyromonas gingivalis Infection Induces Amyloid-B Accumulation in Monocytes/Macrophages. J Alzheimers Dis. 72, 479-494, 2019.

Non-Patent Document No. 10: Mayrand D, Holt S C., Biology of asaccharolytic black-pigmented Bacteroides species. Microbiol Rev. 52, 134-152, 1988.

Non-Patent Document No. 11: Shi Y, Ratnayake D B, Okamoto K et al., Genetic Analyses of Proteolysis, Hemoglobin Binding, and Hemagglutination of Porphyromonas gingivalis. CONSTRUCTION OF MUTANTS WITH A COMBINATION OF rgpA, rgpB, kgp, AND hagA. J Biol Chem. 274, 17955-17960, 1999.

Non-Patent Document No. 12: Imamura T, Potempa J, Travis J., Comparison of pathogenic properties between two types of arginine-specific cysteine proteinases (gingipains-R) from Porphyromonas gingivalis. Microb Pathog. 29, 155-163, 2000.

Non-Patent Document No. 13: Kadowaki T, Takii R, Baba A et al., Gingipains as the determinants of periodontopathogenicity: Folia Pharmacol Jpn. 122, 37-44, 2003.

Non-Patent Document No. 14: Kadowaki T, Kitano S, Baba A et al., Isolation and Characterization of a Novel and Potent Inhibitor of Arg-Gingipain from Streptomyces sp. Strain FA-70, Biol Chem. 384, 911-920, 2003.

Non-Patent Document No. 15: Hara K, Nakao K, and Kawaguchi S, Efficacy of Heat-Killed Lactic Acid Bacterium Enterococcus faecalis on Methicillin-Resistant Staphylococcus aureus (MRSA)-Infected Mice, Journal of New Remedies & Clinics 2018:67 (1): 35-44.

Non-Patent Document No. 16: Nakao K, Hara K, Matsuo T, and Kawaguchi S, Effect of Administration of Heat-Killed Enterococcus faecalis on Infected Animals, Journal of New Remedies & Clinics 2020; 69 (3): 276-8.

Non-Patent Document No. 17: Matsuo T, Nakao K, Hara K, and Kawaguchi S, The Effect of A Heat-Killed Lactic Acid Bacterium Enterococcus faecalis for Inhibiting the Proliferation of Clostridioides (Clostridium) difficile, Jpn. Pharmacol. Ther. 2020; 48 (4): 721-25.

### DISCLOSURE OF THE INVENTION

### Problem for Solution by the Invention

It is an object of the present invention to provide a prophylactic and/or therapeutic agent for P. gingivalis infection.

### Means to Solve the Problem

The present inventor has found that it is possible to inhibit in vitro the growth and the gingipain activity of P. gingivalis using HkEf (Heat-killed lactic acid bacterium Enterococcus faecalis). The present invention has been achieved based on this finding.

The gist of the present invention is as follows.

(1) A prophylactic and/or therapeutic agent for Porphyromonas gingivalis infection, comprising killed Enterococcus faecalis.

(2) A prophylactic and/or therapeutic agent for a disease attributable to Porphyromonas gingivalis, comprising killed Enterococcus faecalis.

(3) A medicine or food for prevention and/or treatment of Porphyromonas gingivalis infection, comprising killed Enterococcus faecalis.

(4) A medicine or food for prevention and/or treatment of a disease attributable to Porphyromonas gingivalis, comprising killed Enterococcus faecalis.

(5) A growth inhibitor for Porphyromonas gingivalis, comprising killed Enterococcus faecalis.

(6) An inhibitor for gingipain activity, comprising killed Enterococcus faecalis.

(7) A method for prevention and/or treatment of Porphyromonas gingivalis infection, comprising administering to a subject a pharmaceutically effective amount of killed Enterococcus faecalis.

(8) Use of killed Enterococcus faecalis for prevention and/or treatment of Porphyromonas gingivalis infection.

(9) Killed Enterococcus faecalis for use in a method for prevention and/or treatment of Porphyromonas gingivalis infection.

(10) A method for prevention and/or treatment of a disease attributable to Porphyromonas gingivalis, comprising administering to a subject a pharmaceutically effective amount of killed Enterococcus faecalis.

(11) Use of killed Enterococcus faecalis for prevention and/or treatment of a disease attributable to Porphyromonas gingivalis.

(12) Killed Enterococcus faecalis for use in a method for prevention and/or treatment of a disease attributable to Porphyromonas gingivalis.

(13) A method for inhibition of the growth of Porphyromonas gingivalis, comprising administering to a subject a pharmaceutically effective amount of killed Enterococcus faecalis.

(14) Use of killed Enterococcus faecalis for inhibition of the growth of Porphyromonas gingivalis.

(15) Killed Enterococcus faecalis for use in a method for inhibition of the growth of Porphyromonas gingivalis.

(16) A method for inhibition of gingipain activity, comprising administering to a subject a pharmaceutically effective amount of killed Enterococcus faecalis.

(17) Use of killed Enterococcus faecalis for inhibition of gingipain activity.

(18) Killed Enterococcus faecalis for use in a method for inhibition of gingipain activity.

### Effect of the Invention

The present invention enables inhibition of the growth of Porphyromonas gingivalis.

The present specification encompasses the contents of the specification and/or drawings disclosed in Japanese Patent Application No. 2021-139391 based on which the present application claims priority.

Further, the present invention enables inhibition of gingipain activity.

### BRIEF DESCRIPTION OF THE DRAWINGS

FIG. 1 shows test results (viable cell count (× 10³ CFU/ml) in Example 1. ∘: Control group: ϵ: Low concentration group (test substance concentration: 1.63 mg/ml); Δ: high concentration group (test substance concentration: 163 mg/ml). Significant difference compared with the control group (**: p<0.01).

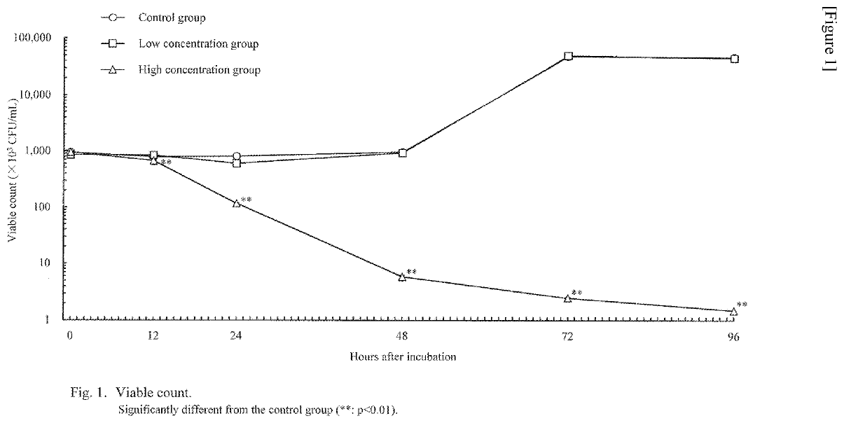

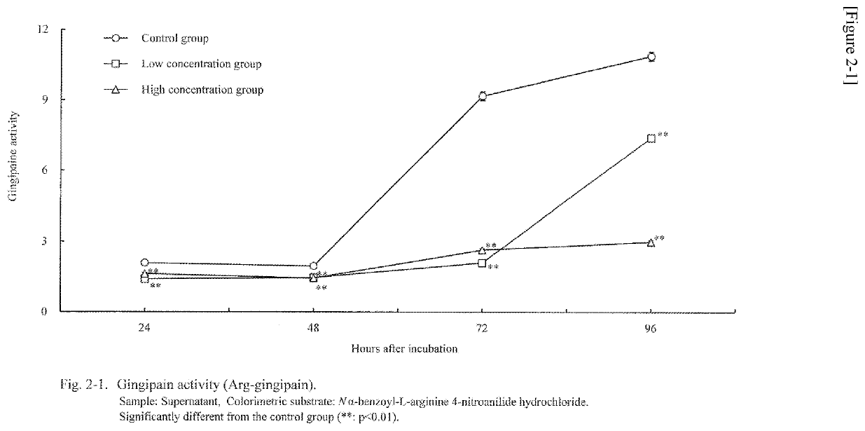

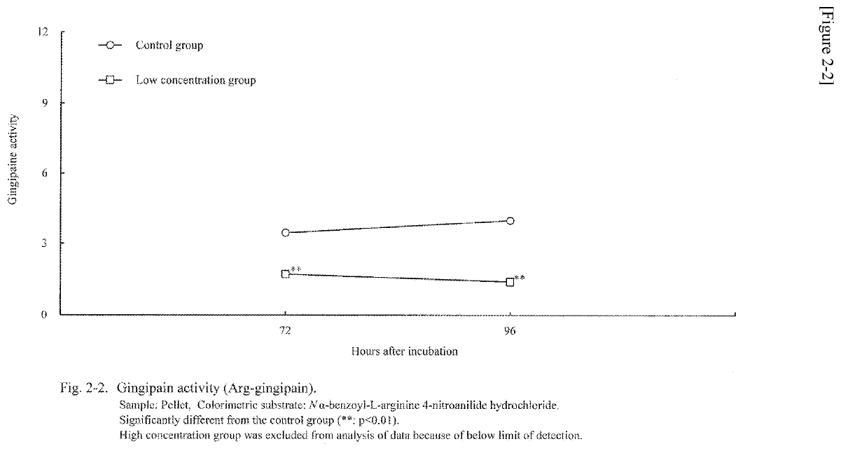

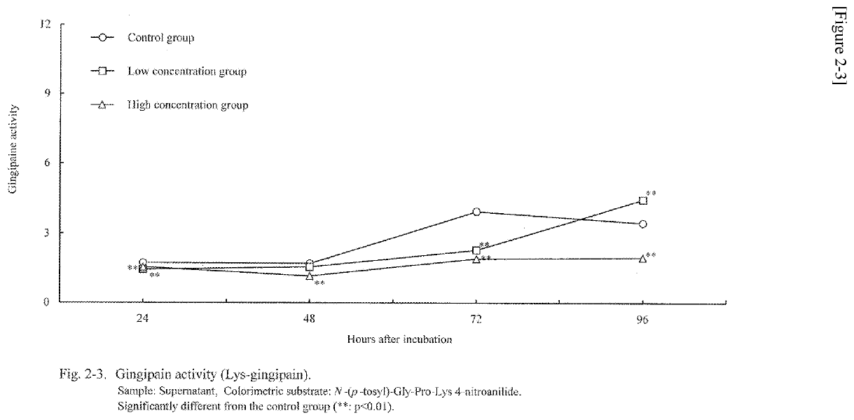

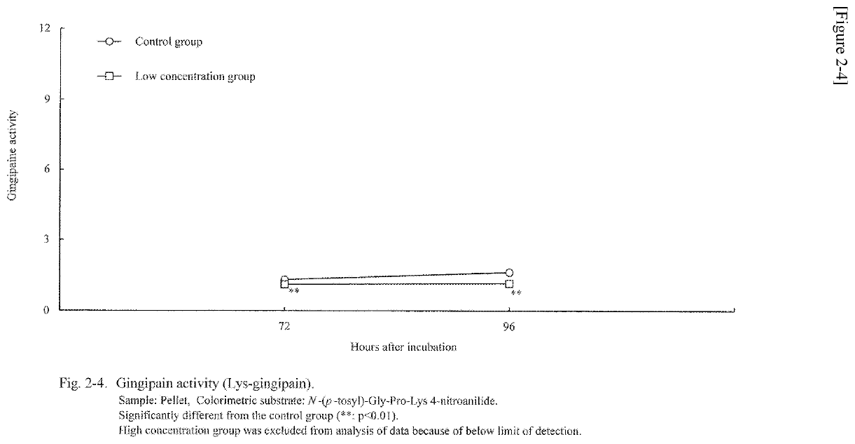

FIG. 2-1 shows test results (gingipain activity (Arg-gingipain) of the supernatant) in Example 1. Colorimetric substrate: Nα-benzoyl-L-arginine 4-nitroanilide hydrochloride.
∘: Control group: □: Low concentration group (test substance concentration: 1.63 mg/ml); Δ: high concentration group (test substance concentration: 163 mg/ml). Significant difference compared with the control group (**: p<0.01).

FIG. 2-2 shows test results (gingipain activity (Arg-gingipain) of the precipitate) in Example 1. Colorimetric substrate: Nα-benzoyl-L-arginine 4-nitroanilide hydrochloride.
∘: Control group: □: Low concentration group (test substance concentration: 1.63 mg/ml). Significant difference compared with the control group (**: p<0.01).

FIG. 2-3 shows test results (gingipain activity (Lys-gingipain) of the supernatant) in Example 1. Colorimetric substrate: N-(p-tosyl)-Gly-Pro-Lys 4-nitroanilide.: ∘: Control group: □: Low concentration group (test substance concentration: 1.63 mg/ml): Δ: high concentration group (test substance concentration: 163 mg/ml). Significant difference compared with the control group (**: p<0.01).

FIG. 2-4 shows test results (gingipain activity (Lys-gingipain) of the precipitate) in Example 1. Colorimetric substrate: N-(p-tosyl)-Gly-Pro-Lys 4-nitroanilide. ∘: Control group: □: Low concentration group (test substance concentration: 1.63 mg/ml). Significant difference compared with the control group (**: p<0.01).

### BEST MODES FOR CARRYING OUT THE INVENTION

Hereinafter, embodiments of the present invention will be described in more detail.

The present invention provides a prophylactic and/or therapeutic agent for P. gingivalis infection, comprising killed Enterococcus faecalis. The present invention also provides a method for prevention and/or treatment of P. gingivalis infection, comprising administering to a subject a pharmaceutically effective amount of killed Enterococcus faecalis. Further, the present invention provides use of killed Enterococcus faecalis for prevention and/or treatment of P. gingivalis infection: or killed Enterococcus faecalis for use in a method for prevention and/or treatment of P. gingivalis infection.

Still further, the present invention provides a prophylactic and/or therapeutic agent for a disease attributable to P. gingivalis, comprising killed Enterococcus faecalis. The present invention provides a method for prevention and/or treatment of a disease attributable to P. gingivalis, comprising administering to a subject a pharmaceutically effective amount of killed Enterococcus faecalis. The present invention also provides use of killed Enterococcus faecalis for prevention and/or treatment of a disease attributable to P. gingivalis; or killed Enterococcus faecalis for use in a method for prevention and/or treatment of a disease attributable to P. gingivalis.

Still further, the present invention provides a growth inhibitor for P. gingivalis, comprising killed Enterococcus faecalis. The present invention provides a method for inhibition of the growth of P. gingivalis, comprising administering to a subject a pharmaceutically effective amount of killed Enterococcus faecalis. The present invention provides use of killed Enterococcus faecalis for inhibition of the growth of P. gingivalis; or killed Enterococcus faecalis for use in a method for inhibition of the growth of P. gingivalis.

Still further, the present invention provides an inhibitor for gingipain activity, comprising killed Enterococcus faecalis. The present invention provides a method for inhibition of gingipain activity, comprising administering to a subject a pharmaceutically effective amount of killed Enterococcus faecalis. The present invention also provides use of killed Enterococcus faecalis for inhibition of gingipain activity: or killed Enterococcus faecalis for use in a method for inhibition of gingipain activity.

Enterococcus faecalis is known as a lactic acid coccal bacterium having biological response modifier (BRM) activity (YAKUGAKU ZASSHI, 112:919-925, 1992; YAKUGAKU ZASSHI, 113:396-399, 1992; Journal of Animal Clinical Research, 3:11-20, 1994). Enterococcus faecalis EF-2001 strain is available from Nihon Berumu Co., Ltd. (2-14-3Nagatacho, Chiyoda-ku, Tokyo).

Enterococcus faecalis-2001 strain can be obtained from the stool of a normal person and has the following properties.

A Gram-positive coccus. Shape of colony (Trypto-Soya agar medium, 24-hour culture): 1.0-mm diameter, smooth, precise circle, white colony. Bacterial morphology: circular to oval (1.0×1.5 μm). Likely to form chains in liquid media. Non-spore-forming. Facultative anaerobic. Ferments glucose to produce lactic acid (final pH: 4.3). Non-gas-producing. Catalase-negative. Proliferates at 10 to 45° C. (the optimal temperature is 37° C.). Proliferates to pH 9.6, 6.5% NaCl, and 40% bile. Positive for 0.04% potassium tellurite. Positive for 0.01% tetrazolium. Positive for 0.1% methylene blue milk. Hydrolyzes arginine. Ferments amygdalin, cellobiose, fructose, galactose, glucose, glycerol, lactose, maltose, mannose, mannitol, ribose, salicin, sucrose, melicitose, and sorbitol to produce acids. Resistant at 60° C. for 30 minutes. Digests casein and gelatin. Decarboxylates tyrosine into tyramine. Lancefield antigen group: D. GC %: 35.0±1.0%.

Enterococcus faecalis may suitably be a killed bacterium, and the bacterium may be subjected to a destruction treatment (e.g., homogenization, enzyme treatment, or ultrasonication) or any other treatment such as heating or drying (e.g., freeze-drying or spray-drying). Viable Enterococcus faecalis may be killed by heating. Killed Enterococcus faecalis is expected to exhibit an intestinal immunity-activating effect. The particle size of the bacterial cell is typically 0.05 μm-50 μm, preferably 0.08-20 μm, more preferably 0.1-10 μm. The bacterium may be mixed with a diluent, and then a thickener may be added to form granules. It is recommended to select the diluent and thickener from materials approved for addition to foods and medicines.

Killed Enterococcus faecalis acts on the growth of P. gingivalis in a suppressive manner.

P. gingivalis is a gram negative, obligate anaerobe and is a representative pathogenic bacterium which is believed to be most largely involved in the onset and the progression of periodontitis. It has been pointed out that P. gingivalis is associated with not only oral diseases but also various systemic diseases such as cardiovascular diseases (endocarditis, coronary heart disease, etc.), pneumonia, preterm delivery and delivery of low weight babies.

P. gingivalis produces gingipain, a protease, on its cell surfaces or extracellularly. Killed Enterococcus faecalis acts on gingipain activity in a suppressive manner. There are two types of gingipains different in the specificity of peptide cleavage site: Arg-gingipain (Rgp) and Lys-gingipain (Kgp). Killed Enterococcus faecalis is capable of inhibiting the activities of both Rgp and Kgp.

Acting on the growth of P. gingivalis and gingipain activity in a suppressive manner, killed Enterococcus faecalis may be used for prevention and/or treatment of P. gingivalis infection (e.g., periodontitis). Further, killed Enterococcus faecalis may be used for prevention and/or treatment of a disease attributable to P. gingivalis. Specific examples of P. gingivalis infection include, but are not limited to, periodontitis and dementia (such as Alzheimer's disease). Specific examples of diseases attributable to P. gingivalis include, but are not limited to, cardiovascular diseases (such as endocarditis, coronary heart disease, etc.), pneumonia, preterm delivery, delivery of low weight babies, atherosclerosis, dementia (such as Alzheimer's disease), rheumatoid arthritis, obesity, non-alcoholic steatohepatitis (NASH), diabetes, frailty and sarcopenia. The prophylactic and/or therapeutic agent of the present invention which is either for P. gingivalis infection or for a disease attributable to P. gingivalis may be used as a medicine or a food additive. The present invention provides a medicine or food for prevention and/or treatment of P. gingivalis infection, comprising killed Enterococcus faecalis. The present invention also provides a medicine or food for prevention and/or treatment of a disease attributable to P. gingivalis, comprising killed Enterococcus faecalis.

When the agent is used as a medicine, it is recommended that killed Enterococcus faecalis be used alone or be mixed with an excipient or a carrier to make a formulation such as a tablet, a capsule, a powder, a granule, a liquid, a syrup, an aerosol, a cream, a gel, an ointment, a gargle, a suppository, or an injection. The excipient or carrier may be any excipient or carrier that is commonly used in the art and is pharmaceutically acceptable, and the type and composition of the excipient or carrier are chosen as appropriate. For example, water or a vegetable oil is used as a liquid carrier. As a solid carrier, there is used, for example, a sugar such as lactose, sucrose, or glucose, a starch such as potato starch or corn starch, or a cellulose derivative such as crystalline cellulose. A lubricant such as magnesium stearate, a binder such as gelatin or hydroxypropyl cellulose, and a disintegrant such as carboxymethyl cellulose may also be added. As bases which may be used for agents for external application, a hydrophobic base such as fats & oils, waxes or hydrocarbons, a water-soluble base such as macrogol, an emulsified base in which oil and water are emulsified with a surfactant, a hydrogel base, a lyogel base, or the like may be enumerated. Further, an antioxidant, a colorant, a flavoring agent, a preservative, or the like may also be added. The medicine can also be used as a freeze-dried formulation.

The medicine of the present invention can be administered by various routes, such as orally, nasally, rectally, transdermally, subcutaneously, intravenously, and intramuscularly. The medicine of the present invention may be applied to the oral cavity.

The content of killed Enterococcus faecalis in the formulation varies depending on the type of the formulation, and is typically 0.001 to 100% by mass and preferably 0.01 to 100% by mass.

The dose of killed Enterococcus faecalis may be any pharmaceutically effective amount, and it varies depending on the form of the formulation, the administration route, the age and body weight of the patient, the severity of the disease, and the like. In the case of an adult patient, for example, it is recommended to set the dose per administration to about 100,000,000 to 100,000,000,000 CFU/kg body weight, preferably about 1,000,000,000 to 50,000,000,000 CFU/kg body weight, and more preferably about 6,000,000,000 to 20,000,000,000 CFU/kg body weight, in terms of the amount of killed Enterococcus faecalis, and to give one to several (e.g., 2, 3, 4, or 5) administrations per day. Administration period is not particularly limited and may, for example, be 7 days or more, 10 days or more, or 17 days or more.

Killed Enterococcus faecalis may be added to a food. The following may be added to the food of the present invention: general ingredients such as protein, fat, carbohydrate, and sodium; minerals such as potassium, calcium, magnesium, and phosphorus; trace elements such as iron, zinc, copper, selenium, and chromium; vitamins such as vitamin A, β-carotene, vitamin B1, vitamin B2, vitamin B6, vitamin B12, vitamin C, niacin, folic acid, vitamin D3,vitamin E, biotin, and pantothenic acid; and other substances such as coenzyme Q10, α-lipoic acid, galacto-oligosaccharide, dietary fiber, an excipient (such as water, carboxymethyl cellulose, or lactose), a sweetener, a flavoring agent (such as malic acid, citric acid, or amino acid), and a fragrance. When the food of the present invention is provided as a liquid food, water, saline solution, fruit juice, or the like can be used as a liquid in which the food ingredients are dispersed or dissolved. In order to improve the taste in oral administration, it is recommended to use fruit juice. The food of the present invention may be formulated into any form such as a powder, a granule, a tablet, or a liquid. In order to allow sick or old persons to easily take the food, it is preferable that the food be a gelled product such as jelly.

Gelling agents that can be used include thickening polysaccharides such as dextrin, agar, xanthan gum, locust bean gum, carrageenan, and pectin; gellan gum, psyllium seed gum, tara gum, guar gum, glucomannan, alginic acid, tamarind seed gum, and cellulose; and it is preferable to use one or two or more thickening polysaccharides. As regards the gel strength of the gelled product, it is preferable that the gel strength at 5° C. be 7,000±2,000 N/m². When the gel strength is 7,000±2,000 N/m², it is more preferable that the adhesion energy be 60±40 J/m³ and the cohesiveness be 0.7±0.1 J/m³. Such a gel with low adhesiveness and high cohesiveness has excellent swallowability.

The gel strength can be measured as follows. A texturometer of YAMADEN Co., Ltd. and a 16-mm-diameter plunger are used as gel strength measurement instruments, and the measurement is carried out under the following conditions: the measurement temperature is 25° C., the compression speed (the speed at which the plunger is pushed in) is 10 mm/s, the measurement strain (the ratio of the amount of pushing-in to the sample thickness) is 40.00%, the distance over which the plunger is pushed in is 10.00 mm, and the number of repetitions of pushing-in of the plunger is two.

The adhesion energy is measured as a negative energy required for pulling out the plunger after the first pushing-in of the plunger in the above gel strength measurement.

The cohesiveness is measured as the ratio between the energy at the first pushing-in and the energy at the second pushing-in in the above gel strength measurement.

The food to which killed Enterococcus faecalis can be added may be chewing gum, candy, gummy candy, throat lozenge or the like.

The intake of killed Enterococcus faecalis may be any amount that is sufficient to achieve the purpose of the food, and it varies depending on the form of the formulation, the administration route, the age and body weight of the patient, the severity of the disease, and the like. In the case of an adult patient, for example, it is recommended to set the dose per administration to about 100,000,000 to 100,000,000,000 CFU/kg body weight, preferably about 1,000,000,000 to 50,000,000,000 CFU/kg body weight, and more preferably about 6,000,000,000 to 20,000,000,000 CFU/kg body weight, in terms of the amount of killed Enterococcus faecalis, and to give one to several (e.g., about 2, 3, 4, or 5) administrations per day.

### EXAMPLES

Hereinafter, the present invention will be described in detail based on the Example below.

### Example 1

### Test to Confirm the Action of Lactic Acid Bacterium EF-2001 Strain on P. gingivalis

### (Materials and Methods)

### Test Substance

### Test Substance

Name: LACTIC ACID BACTERIA POWDER EF-2001 (Nihon Berumu Co., Ltd.)
Properties: Yellow-brown powder
Storage conditions: Room temperature (18.0-28.0° C.), light-shielded, moisture-proof Administration Sample

### Preparation of Test Substance

The necessary amount of lactic acid bacteria powder EF-2001 was weighed (electronic balance: XP205DR, Mettler-Toledo Co., Ltd.) and suspended in Brain-Heart Infusion Broth to prepare a suspension of 500 mg/ml. This 500 mg/ml suspension was serially diluted with Brain-Heart Infusion Broth to give concentrations of 163 mg/ml and 1.63 mg/ml. Since the lactic acid bacteria powder precipitates, it was stirred well enough to be kept suspended. Preparation was made just before use.

### Pathogenic Microorganism

### Strain Used

Porphyromonas gingivalis (ATCC 33277)

### Storage Conditions

Cryopreserved in an Ultra-cold freezer (controlled temperature: −80° C., MDF-394AT, Sanyo Electric Co., Ltd.) until use.

### Reagents

(1) Brucella HK Agar Medium (Kyokuto Pharmaceutical Industrial Co., Ltd)
(2) Brain-Heart Infusion Broth (EIKEN Chemical Co., Ltd.)
(3) Physiological saline (Otsuka Pharmaceutical Plant, Inc.)

### Preculture

The preserved strain of P. gingivalis was thawed and smeared on Brucella HK Agar Medium. The resultant medium was transferred into a deoxidant-filled anaerobic jar, and then cultured for 3 days in an incubator (ILE800, Yamato Scientific Co., Ltd.) set at 37° C. After culture, colonies were harvested and inoculated to Brain-Heart Infusion Broth (containing 5 μg/ml hemin and 1 μg/ml menadione), which was transferred into a deoxidant-filled anaerobic jar and cultured anaerobically for 2 days in an incubator (ILE800, Yamato Scientific Co., Ltd.) set at 37° C. to give a turbidity (OD₆₅₀) of 0.6. The resultant culture broth served as an inoculation stock solution.

### Preparation of a Liquid Bacterial Inoculum and Taking of Viable Cell Count

The stock solution diluted 10⁴-folds with Brain-Heart Infusion Broth was used as a liquid bacterial inoculum.

Confirmation of viable cell count was performed as follows. Briefly, an aliquot of liquid bacterial inoculum was taken, diluted 10-folds, 10²-folds or 10³-folds with physiological saline, and smeared on Brucella HK Agar Medium. Then, the medium was transferred into a deoxidant-filled anaerobic jar and cultured for 4 days in an incubator set at 37° C. The number of colonies after culture was counted with a handy colony counter (CC-1, Azwan Co., Ltd.), and the number of viable bacterial cells contained in 1 ml of the inoculation stock solution was calculated.

### Testing Method

1. Growth Inhibition Test

### Description of Test Groups

| | | Concentration | Concentration of |
|---|---|---|---|
| | | of Test | P. gingivalis |
| Group | | Substance | Inoculum |
| No. | Test Group | (mg/ml) | (CFU/ml) |
| 1 | Control | 0* | 1.2 × 10⁶ |
| 2 | Low concentration** | 0.64 | 1.2 × 10⁶ |
| 4 | High concentration | 64 | 1.2 × 10⁶ |

Indicated are the final concentrations after mixing.
*Brain-Heart Infusion Broth was added.
**Low concentration group is a model in which P. gingivalis was added to a product 80 mg/125 ml (600,000,000,000 cells of lactic acid bacterium being present in 80 mg).

### Confirmation of Viable Cell Count

To 1 ml of bacterial inoculum in each test tube, 0.65 ml of the above-prepared sample suspension (1.63 mg/ml or 163 mg/ml) was added. Then, test tubes were placed in a deoxidant-filled anaerobic jar, and P. gingivalis was cultured in an incubator set at 37° C. Each test tube was taken out at the beginning of culture and at 12, 24, 48, 72 and 96 hours of culture, and pH of the culture broth was measured. Then, an aliquot of the culture broth was collected for taking the viable cell count.

The culture broth for taking of viable cell count was appropriately diluted. Undiluted and diluted culture broths were smeared on Brucella HK Agar Medium, which was then anaerobically cultured for 4 days in an incubator set at 37° C. The number of colonies after culture was counted with a handy colony counter, and the number of viable bacterial cells was calculated. The number of samples was 5.

Test tubes from which an aliquot of culture broth had been collected for taking the viable cell count were centrifuged in a centrifuge (AX-310, TOMY SEIKO CO., LTD.) (2000 rpm, 10 min.). The supernatant was collected, and the precipitate was suspended in Tris-HCl buffer. Gingipain activities of the supernatant and the precipitate were measured. The number of each sample was 5.

### Measurement of Gingipain Activity¹⁾

A reaction solution 100 μl in total which was composed of the supernatant or the precipitate suspension, 50 mM Tris-HCl buffer (pH 8.5), 0.2 mM colorimetric substrate (Nα-benzoyl-L-arginine 4-nitroanilide hydrochloride for Arg-gingipain, or N-(p-tosyl)-Gly-Pro-Lys 4-nitroanilide for Lys-gingipain), and 10 mM DTT was subjected to reaction at 37° C. for 10 minutes. Then, reaction was terminated by adding 25 μl of 50% acetic acid. Absorbance was measured with a plate reader (Multiskan™ FC, Thermo Fisher Scientific) at a wavelength of 405 nm. The supernatant and the precipitate_suspension were measured for absorbance with the plate reader at a wavelength of 620 nm. Gingipain activity was calculated through dividing the absorbance at a wavelength of 405 nm by the value at a wavelength of 620 nm.

### (Literature)

1) Masuda T, et al., Effects of various growth conditions in a chemostat on expression of virulence factors in Porphyromonas gingivalis. Appl. Environ. Microbiol. 72.2006 3458-3467.

### Summary of Results

As regards viable cell count and gingipain activity, mean and standard error were calculated.

For significance test, Wilcoxon rank sum test was used for control group vs test groups on viable cell count and gingipain activity.

A hazard rate of 5% was considered significant, and separate indications were given for a hazard rate less than 5% and a hazard rate less than 1%.

A commercially available statistical program (SAS system; SAS Institute Japan) was used for the statistical analyses.

### Test Results

### 1. Viable Cell Count

Test results are shown in Table 1 and FIG. 1.

The viable cell count of P. gingivalis in the control group was 942.0±23.5 (×10³ CFU/ml) at the beginning of culture, 784.0±11.2 (×10³ CFU/ml) at 12 hours of culture, 810.0±55.9 (×10³ CFU/ml) at 24 hours of culture, 960.0±34.2 (×10³ CFU/ml) at 48 hours of culture, 48380.0±3239.4 (×10³ CFU/ml) at 72 hours of culture, and 46800.0±3253.0 (×10³ CFU/ml) at 96 hours of culture.

In the low concentration group, the viable cell count of P. gingivalis changed at similar levels to the control group.

In the high concentration group, a significant decrease in the viable cell count of P. gingivalis was observed from 12 to 96 hours of culture, as compared to the control group.

### 2. Gingipain Activity

Test results are shown in Tables 2-1 through 2-4 and FIGS. 2-1 through 2-4. As blank value, test results of gingipain activity obtained from vehicle alone (consisting of test substance i.e., lactic acid bacterium, culture broth, and colorimetric substrate) are shown in Attachment 1 (Table 3).

Since the vehicle used in this test (neither the supernatant nor precipitate_suspension but consisting of culture broth, test substance and colorimetric substrate alone) showed absorbance at the same wavelength as that of gingipain activity, gingipain activity was measured immediately after addition of culture broth, test substance and colorimetric substance. As it turned out, the maximum value of gingipain activity was 0.98.

Therefore, values less than 1.0 were regarded as below detection limit in this test. For evaluation, mean values of 1.0 and more were used (FIGS. 2-1 through 2-4).

As a result, the gingipain activity of the control group began to increase at 24 hours of culture in the supernatant and at 72 hours of culture in the precipitate with respect to Nα-benzoyl-L-arginine 4-nitroanilide hydrochloride or N-(p-tosyl)-Gly-Pro-Lys 4-nitroanilide, whereas the gingipain activities of the low and high concentration groups as compared to the control group showed small increases with respect to both substrates, thus showing significantly low values of gingipain activity.

### 3. pH Measurement of Culture Broth

Measurement results are shown in Attachment 2 (Table 4).

The pH of culture broth in the high concentration group tended to show low values. When the control group and the high concentration group were compared, there was a difference of approximately 0.8 at maximum.

TABLE 1
Viable count

| | Group | | |
|---|---|---|---|
| | | Low | High |
| | Control | concentration | concentration |
| | Number of samples | | |
| | 5 | 5 | 5 |
| Hours after | | | |
| incubation | | | |
| 0 | 942.0 ± 23.5 | 854.0 ± 38.8 | 944.0 ± 26.2 |
| 12 | 784.0 ± 11.2 | 838.0 ± 20.1 | 670.0 ± 17.3 ** |
| 24 | 810.0 ± 55.9 | 602.0 ± 52.0 | 117.8 ± 2.4 ** |
| 48 | 960.0 ± 34.2 | 928.0 ± 30.1 | 5.8 ± 0.4 ** |
| 72 | 48380.0 ± 3239.4 | 49960.0 ± 3498.4 | 2.5 ± 0.1 ** |
| 96 | 46800.0 ± 3253.0 | 45640.0 ± 3224.4 | 1.5 ± 0.0 ** |

Each value shows mean (×10³ CFU/mL) ± S.E.
Significantly different from the control group (**: p < 0.01).

### (Table 2-1)

TABLE 2-1
Gingipain activity (Arg-gingipain)

| | Group | | |
|---|---|---|---|
| | | Low | High |
| | Control | concentration | concentration |
| | Number of samples | | |
| | 5 | 5 | 5 |
| Hours after | | | |
| incubation | | | |
| 0 | 0.49 ± 0.01 a) | 0.46 ± 0.00 a) | 0.63 ± 0.01 a) |
| 12 | 0.59 ± 0.01 a) | 0.61 ± 0.04 a) | 0.59 ± 0.03 a) |
| 24 | 2.08 ± 0.05 | 1.39 ± 0.03 ** | 1.61 ± 0.05 ** |
| 48 | 1.95 ± 0.03 | 1.45 ± 0.03 ** | 1.43 ± 0.01 ** |
| 72 | 9.18 ± 0.18 | 2.09 ± 0.02 ** | 2.64 ± 0.06 ** |
| 96 | 10.89 ± 0.19 | 7.42 ± 0.15 ** | 2.99 ± 0.07 ** |

Each value shows mean ± S.E.
Sample: Supernatant.
Colorimetric substrate: N α-benzoyl-L-arginine 4-nitroanilide hydrochloride.
a): Excluded from analysis of data because of below limit of detection.
Significantly different from the control group (**: p < 0.01).

TABLE 2-2
Gingipain activity (Arg-gingipain)

| | Group | | |
|---|---|---|---|
| | | Low | High |
| | Control | concentration | concentration |
| | Number of samples | | |
| | 5 | 5 | 5 |
| Hours after | | | |
| incubation | | | |
| 0 | 0.59 ± 0.01 a) | 0.56 ± 0.00 a) | 0.85 ± 0.00 a) |
| 12 | 0.53 ± 0.03 a) | 0.67 ± 0.00 a) | 0.84 ± 0.00 a) |
| 24 | 0.51 ± 0.02 a) | 0.67 ± 0.00 a) | 0.99 ± 0.01 a) |
| 48 | 0.47 ± 0.01 a) | 0.87 ± 0.00 a) | 0.84 ± 0.00 a) |
| 72 | 3.46 ± 0.01 | 1.71 ± 0.00 ** | 0.88 ± 0.00 a) |
| 96 | 3.99 ± 0.02 | 1.40 ± 0.02 ** | 0.84 ± 0.01 a) |

Each value shows mean ± S.E.
Sample: Pellet.
Colorimetric substrate: N α-benzoyl-L-arginine 4-nitroanilide hydrochloride.
a): Excluded from analysis of data because of below limit of detection.
Significantly different from the control group (**: p < 0.01).

TABLE 2-3
Gingipain activity (Lys-gingipain)

| | Group | | |
|---|---|---|---|
| | | Low | High |
| | Control | concentration | concentration |
| | Number of samples | | |
| | 5 | 5 | 5 |
| Hours after | | | |
| incubation | | | |
| 0 | 0.54 ± 0.01 a) | 0.44 ± 0.01 a) | 0.56 ± 0.01 a) |
| 12 | 0.53 ± 0.02 a) | 0.70 ± 0.03 a) | 0.51 ± 0.02 a) |
| 24 | 1.72 ± 0.01 | 1.43 ± 0.03 ** | 1.53 ± 0.01 ** |
| 48 | 1.69 ± 0.06 | 1.54 ± 0.02 | 1.15 ± 0.01 ** |
| 72 | 3.94 ± 0.14 | 2.28 ± 0.02 ** | 1.90 ± 0.03 ** |
| 96 | 3.45 ± 0.07 | 4.44 ± 0.09 ** | 1.95 ± 0.05 ** |

Each value shows mean ± S.E.
Sample: Supernatant.
Colorimetric substrate: N-(p-tosyl)-Gly-Pro-Lys 4-nitroanilide
a): Excluded from analysis of data because of below limit of detection.
Significantly different from the control group (**: p < 0.01).

### (Table 2-4)

TABLE 2-4
Gingipain activity (Lys-gingipain)

| | Group | | |
|---|---|---|---|
| | | Low | High |
| | Control | concentration | concentration |
| | Number of samples | | |
| | 5 | 5 | 5 |
| Hours after | | | |
| incubation | | | |
| 0 | 0.53 ± 0.01 a) | 0.63 ± 0.00 a) | 0.87 ± 0.00 a) |
| 12 | 0.40 ± 0.02 a) | 0.68 ± 0.01 a) | 1.01 ± 0.01 |
| 24 | 0.38 ± 0.01 a) | 0.67 ± 0.00 a) | 0.89 ± 0.00 a) |
| 48 | 0.37 ± 0.01 a) | 0.80 ± 0.00 a) | 0.93 ± 0.01 a) |
| 72 | 1.33 ± 0.01 | 1.13 ± 0.01 ** | 0.89 ± 0.00 a) |
| 96 | 1.64 ± 0.01 | 1.17 ± 0.01 ** | 0.87 ± 0.00 a) |

Each value shows mean ± S.E.
Sample: Pellet
Colorimetric substrate: N-(p-tosyl)-Gly-Pro-Lys 4-nitroanilide.
a): Excluded from analysis of data because of below limit of detection.
Significantly different from the control group (**: p < 0.01).

TABLE 3
Attachment 1. Gingipain activity

| | Nα-benzoyl-L-arginine 4-nitroanilide hydrochloride | | | | | | N-(p-tosyl)-Gly-Pro-Lys 4-nitroanilide | | | | | |
|---|---|---|---|---|---|---|---|---|---|---|---|---|
| | Control | | Low concentration | | High concentration | | Control | | Low concentration | | High concentration | |
| Sample No. | Pellet | Supernatant | Pellet | Supernatant | Pellet | Supernatant | Pellet | Supernatant | Pellet | Supernatant | Pellet | Supernatant |
| 1 | 0.66 | 0.50 | 0.60 | 0.53 | 0.98 | 0.58 | 0.47 | 0.53 | 0.58 | 0.49 | 0.92 | 0.51 |
| 2 | 0.56 | 0.54 | 0.58 | 0.54 | 0.98 | 0.58 | 0.53 | 0.52 | 0.61 | 0.49 | 0.94 | 0.54 |
| 3 | 0.54 | 0.49 | 0.60 | 0.49 | 0.95 | 0.59 | 0.53 | 0.57 | 0.63 | 0.50 | 0.93 | 0.46 |
| 4 | 0.64 | 0.56 | 0.60 | 0.53 | 0.97 | 0.52 | 0.50 | 0.58 | 0.63 | 0.49 | 0.96 | 0.49 |
| 5 | 0.61 | 0.52 | 0.61 | 0.54 | 0.98 | 0.57 | 0.54 | 0.54 | 0.60 | 0.48 | 0.94 | 0.50 |
| Number of samples | 5 | 5 | 5 | 5 | 5 | 5 | 5 | 5 | 5 | 5 | 5 | 5 |
| Mean | 0.60 | 0.52 | 0.60 | 0.53 | 0.97 | 0.57 | 0.51 | 0.55 | 0.61 | 0.49 | 0.94 | 0.50 |
| S.E. | 0.02 | 0.01 | 0.00 | 0.01 | 0.01 | 0.01 | 0.01 | 0.01 | 0.01 | 0.00 | 0.01 | 0.01 |

The test substance was added to the culture medium and measured immediately.

TABLE 4
Attachment 2. pH Value

| | | pH Value | | | | | |
|---|---|---|---|---|---|---|---|
| | Test | Start | After | After | After | After | After |
| Test | Tube | of | 12 | 24 | 48 | 72 | 96 |
| Group | No. | culture | hours | hours | hours | hours | hours |
| Control | 1 | 7.40 | 7.35 | 7.33 | 7.07 | 7.03 | 6.86 |
| | 2 | 7.35 | 7.36 | 7.31 | 7.10 | 7.13 | 6.77 |
| | 3 | 7.44 | 7.41 | 7.41 | 7.05 | 7.08 | 6.81 |
| | 4 | 7.46 | 7.47 | 7.38 | 7.09 | 7.14 | 6.74 |
| | 5 | 7.36 | 7.52 | 7.39 | 7.13 | 7.16 | 6.73 |
| Mean | | 7.40 | 7.42 | 7.36 | 7.09 | 7.11 | 6.78 |
| Standard error | | 0.02 | 0.03 | 0.02 | 0.01 | 0.02 | 0.02 |
| Low | 1 | 7.21 | 7.15 | 7.19 | 7.05 | 7.04 | 6.78 |
| concen- | 2 | 7.25 | 7.11 | 7.22 | 7.12 | 7.08 | 6.88 |
| tration | 3 | 7.13 | 7.04 | 7.23 | 7.04 | 7.16 | 6.81 |
| | 4 | 7.07 | 7.03 | 7.17 | 7.09 | 7.18 | 6.62 |
| | 5 | 7.11 | 7.16 | 7.29 | 7.13 | 7.04 | 6.66 |
| Mean | | 7.15 | 7.10 | 7.22 | 7.09 | 7.10 | 6.75 |
| Standard error | | 0.03 | 0.03 | 0.02 | 0.02 | 0.03 | 0.05 |
| High | 1 | 6.91 | 6.88 | 6.86 | 6.31 | 6.58 | 6.33 |
| concen- | 2 | 6.97 | 6.71 | 6.81 | 6.25 | 6.41 | 6.44 |
| tration | 3 | 6.83 | 6.73 | 6.87 | 6.38 | 6.53 | 6.47 |
| | 4 | 6.84 | 6.81 | 6.93 | 6.27 | 6.47 | 6.51 |
| | 5 | 6.88 | 6.92 | 6.97 | 6.42 | 6.32 | 6.54 |

| | | | | | | |
|---|---|---|---|---|---|---|
| Mean | 6.89 | 6.81 | 6.89 | 6.33 | 6.46 | 6.46 |
| Standard error | 0.03 | 0.04 | 0.03 | 0.03 | 0.05 | 0.04 |

### Discussion

In this study, the inhibitory effect of HkEf (heat-killed lactic acid bacterium Enterococcus faecalis) on the growth of P. gingivalis in vitro was examined. Results were as follows. Low concentration HkEf group did not exhibit an inhibitory effect in comparison with the increase of viable cell count of P. gingivalis as seen in the control group, and similar changes were seen in the two groups; on the other hand, in high concentration HkEf group, the increase of viable cell count was inhibited significantly over time compared to the control group, thus showing an inhibitory effect on the growth of P. gingivalis. In contrast, both low and high concentration HkEf groups were shown to exhibit a significant inhibitory effect on the increase of gingipain activities of Rap and Kgp which are toxins produced by P. gingivalis. Thus, a difference was observed between low and high HkEf concentration groups with respect to the growth inhibitory effect of HkEf on P. gingivalis viable cell count but as regards the increase of gingipain activity, even the low concentration group which did not affect the viable cell count of P. gingivalis was shown to have an inhibitory effect on the increase of gingipain activity. In other words, HkEf was confirmed to have an inhibitory effect both on the growth of P. gingivalis and on the increase of gingipain activity.

Previous studies in vitro on the growth inhibitory effect against P. gingivalis of lactic acid bacteria have yielded a report¹⁸⁾ that an extract from a lactic acid bacterium Enterococcus faecium (E. faecium) inhibits the growth of P. gingivalis but this is a study using viable cells of lactic acid bacteria. Further, antimicrobial peptides¹⁹⁾ as well as natural and synthetic gingipain inhibitors²⁰⁾ have also been studied, but problems have been pointed out regarding their specificity, safety and efficacy, as well as high production cost. Although P. gingivalis is a bacterium that is slow in subgingival biofilm formation, gingipain that is a protease produced and extracellularly secreted by P. gingivalis²¹⁾ gives damage to host cells and eliminates the adhesiveness of gingival fibroblasts and vascular endothelial cells, thereby inducing cell death. The two enzymes of gingipain, Rgp and Kgp, cooperate with each other to degrade biological proteins, and both of them degrade extracellular matrices including human collagen types I and IV, fibronectin, and laminin²²⁻²⁴⁾, as well as subunits α2, β1, and β3of integrin, which is a receptor protein for fibronectin¹³⁾. In addition, it has also been revealed that both enzymes inhibit the phagocytosis action of neutrophils since they destroy human immunoglobulins (IgG, IgA) and complement components (C3, C5) and degrade/inactivate cytokines (IL-6, IL-8, TNF-α), thereby impairing the defense mechanism in a living body²⁵⁻²⁸⁾. Therefore, it has become clear that the strong damaging effect which gingipain gives to the host by degrading biological proteins is a risk factor not only for chronic periodontitis but also for various systemic diseases such as cardiovascular diseases, pneumonia and Alzheimer's disease. Considering these findings, the inhibitory effect of HkEf on the activities of two gingipain types Rgp and Kgp as observed in this study is expected to have important meaning in the prevention of various systemic diseases. Further, lactic acid bacteria are known to lower pH by producing organic acids such as lactic acid and acetic acid and inhibit bacterial growth by producing antibacterial substances such as hydrogen peroxide and bacteriocin²⁶⁻²⁸⁾. The lactic acid bacterium HkEf used in this study is a heat-killed bacterium and does not produce lactic acid or the like during its culture process, so the tendency of the culture broth to decrease in pH as observed in high-concentration HkEf group when compared with the control group was thought to be due to a factor other than lactic acid or the like. It should be added here that since the pH change during the culture period was within the range of 6.33 to 6.89 on average (Table 4), the pH of the culture broth would not have greatly affected the inhibitory effect on the growth of P. gingivalis. Since the heat-killed bacterial cells (HkEf) used in the present invention exhibited both an inhibitory effect on the growth of P. gingivalis viable cells and an inhibitory effect on gingipain activity, the antibacterial effect of not viable but heat-killed lactic acid bacterium against P. gingivalis has become clear.

Recent studies have revealed that periodontitis in which P. gingivalis is a pathogenic bacterium increases the risk for diseases such as diabetes^{29) 30)}, arteriosclerotic diseases³¹⁾, rheumatoid arthritis³²⁾, and obesity/non-alcoholic steatohepatitis (NASH)³³⁾. Further, the relation between Alzheimer's disease⁶⁾ and P. gingivalis has been attracting attention. The present study, which has shown that heat-killed bacterium HkEf inhibits the growth of P. gingivalis the involvement of which in many systemic diseases is now becoming clear and that HkEf also inhibits gingipain activity, is believed to have an important significance.

### REFERENCES

1) 2016 Survey of Dental Diseases (Ministry of Health, Labor and Welfare)

2) Maekawa T, Krauss JL, Abe T et al. Porphyromonas gingivalis manipulates complement and TLR signaling to uncouple bacterial clearance from inflammation and promote dysbiosis. Cell Host Microbe. 15:768-778, 2014.

3) Aoyama N, Suda T, Ikeda Y et al., Survey on Systemic Conditions of New Patients Who Visited Periodontitis Outpatient Department of the Tokyo Medical and Dental University Hospital, J. Stomatol. Soc. Jpn., 84, 37-44, 2017.

4) Kimura Y, Shimabara S, Hashimoto N et al., Relationship between Oral Diseases and the Whole Body-Importance of Oral Care, Bulletin of Aino Gakuinn, 27:9-17, 2013.

5) Ilievski V, Zuchowska PK, Green SJ et al. Chronic oral application of a periodontal pathogen results in brain inflammation, neurodegeneration and amyloid beta production in wild type mice. PLOS One. 13, e0204941, 2018.

6) Dominy SS, Lynch C, Ermini F et al. Porphyromonas gingivalis in Alzheimer's disease brains: Evidence for disease causation and treatment with small-molecule inhibitors. Sci Adv. 5: eaau3333, 2019.

7) Poole S, Singhrao SK, Kesavalu L et al. Determining the Presence of Periodontopathic Virulence Factors in Short-Term Postmortem Alzheimer's Disease Brain Tissue. J Alzheimers Dis. 36:665-677, 2013.

8) Poole S, Singhrao SK, Chukkapalli S et al. Active Invasion of Porphyromonas gingivalis and Infection-Induced Complement Activation in ApoE-/-Mice Brains. J Alzheimers Dis. 43, 67-80, 2015.

9) Nie R, Wu Z, Ni J et al. Porphyromonas gingivalis Infection Induces Amyloid-β Accumulation in Monocytes/Macrophages. J Alzheimers Dis. 72, 479-494, 2019.

10) Mayrand D, Holt SC. Biology of asaccharolytic black-pigmented Bacteroides species. Microbiol Rev. 52, 134-152, 1988.

11) Shi Y, Ratnayake DB, Okamoto K et al. Genetic Analyses of Proteolysis, Hemoglobin Binding, and Hemagglutination of Porphyromonas gingivalis. CONSTRUCTION OF MUTANTS WITH A COMBINATION OF rgpA, rgpB, kgp, AND hagA. J Biol Chem. 274, 17955-17960, 1999.

12) Imamura T, Potempa J, Travis J. Comparison of pathogenic properties between two types of arginine-specific cysteine proteinases (gingipains-R) from Porphyromonas gingivalis. Microb Pathog. 29, 155-163, 2000.

13) Kadowaki T, Takii R, Baba A et al., Gingipains as the determinants of periodontopathogenicity. Folia Pharmacol Jpn. 122, 37-44, 2003.

14) Kadowaki T, Kitano S, Baba A et al., Isolation and Characterization of a Novel and Potent Inhibitor of Arg-Gingipain from Streptomyces sp. Strain FA-70, Biol Chem. 384, 911-920, 2003.

15) Hara K, Nakao K, and Kawaguchi S, Efficacy of Heat-Killed Lactic Acid Bacterium Enterococcus faecalis on Methicillin-Resistant Staphylococcus aureus (MRSA)-Infected Mice, Journal of New Remedies & Clinics 2018:67 (1): 35-44

16) Nakao K, Hara K, Matsuo T, and Kawaguchi S, Effect of Administration of Heat-Killed Enterococcus faecalis on Infected Animals, Journal of New Remedies & Clinics 2020:69 (3): 276-87

17) Matsuo T, Nakao K, Hara K, and Kawaguchi S, The Effect of A Heat-Killed Lactic Acid Bacterium Enterococcus faecalis for Inhibiting the Proliferation of Clostridioides (Clostridium) difficile, Jpn. Pharmacol. Ther. 2020; 48 (4): 721-25

18) Ishihara K, Hasegawa A, Mokudai Ret al., Inhibition of the growth of pathogens for caries/periodontitis with enteric Lactobacillus components, The Japanese Journal of Conservative Dentistry, 28:1044-1049, 1985

19) Seo M-D, Won H-S, Kim J-H et al. Antimicrobial Peptides for Therapeutic Applications: A Review: Molecules. 17, 12276-12286, 2012.

20) Kataoka S, Baba A, Suda Y et al. A novel, potent dual inhibitor of Arg-gingipain and Lys-gingipain as a promising agent for periodontal disease therapy. The FASEB Journal. 28, 3564-3578, 2014.

21) Potempa J, Pike R, Travis J. The Multiple Forms of Trypsin-like Activity Present in Various Strains of Porphyromonas gingivalis Are due to the Presence of either Arg-Gingipain or Lys-Gingipain. Infect Immun. 63, 1176-1182, 1995.

22) Kadowaki T, Yoneda M, Okamoto K et al. Purification and characterization of a novel arginine-specific cysteine proteinase (argingipain) involved in the pathogenesis of periodontal disease from the culture supernatant of Porphyromonas gingivalis. J Biol Chem. 269, 21371-21378, 1994.

23) Abe N, Kadowaki T, Okamoto K et al., Biochemical and Functional Properties of Lysine-Specific Cysteine Proteinase (Lys-Gingipain) as a Virulence Factor of Porphyromonas gingivalis in Periodontal Disease. J Biochem. 123, 305-312, 1998.

24) Lantz MS, Allen RD, Duck LW et al. Identification of Porphyromonas gingivalis components that mediate its interactions with fibronectin. J Bacteriol. 173, 4263-4270, 1991.

25) Banbula A, Bugno M, Kuster A, et al., Rapid and Efficient Inactivation of IL-6 Gingipains, Lysine-and Arginine-Specific Proteinases from Porphyromonas Gingivalis. Biochem Biophys Res Commun. 261, 598-602, 1999.

26) Mikolajczyk-Pawlinska J, Travis J, Potempa J, Modulation of interleukin-8 activity by gingipains from Porphyromonas gingivalis: implications for pathogenicity of periodontal disease. FEBS Lett. 440, 282-286, 1998.

27) Calkins CC, Platt K, Potempa J et al., Inactivation of Tumor Necrosis Factor-α by Proteinases (Gingipains) from the Periodontal Pathogen, Porphyromonas gingivalis. IMPLICATIONS OF IMMUNE EVASION. J Biol Chem. 273, 6611-6614, 1998.

28) Wingrove JA, DiScipio RG, Chen Z, et al., Activation of complement components C3 and C5 by a cysteine proteinase (gingipain-1) from Porphyromonas (Bacteroides) gingivalis. J Biol Chem. 267, 18902-18907, 1992.

29) Preshaw PM, Alba AL, Herrera D et al., Periodontitis and diabetes: a two-way relationship. Diabetologia. 55, 21-31, 2012.

30) Arimatsu K et al., Oral pathobiont induces systemic inflammation and metabolic changes associated with alteration of gut microbiota. Sci Rep. 4:4828, 2014.

31) Humphrey LL, Fu R, Buckley DI et al. Periodontal Disease and Coronary Heart Disease Incidence: A Systematic Review and Meta-analysis. J Gen Intern Med. 23:2079-2086, 2008.

32) Sandhya P, Danda D, Sharma D et al. Does the buck stop with the bugs?: an overview of microbial dysbiosis in rheumatoid arthritis. Int J Rheum Dis. 19, 8-20, 2015.

33) Nakajima M et al. Oral pathobiont induces systemic inflammation and metabolic changes associated with alteration of gut microbiota. PLOS One. 7: e0134234, 2015.

All publications, patents and patent applications cited herein are incorporated herein by reference in their entirety.

### INDUSTRIAL APPLICABILITY

The present invention is applicable to prevention and/or treatment of Porphyromonas gingivalis infection.

1-6. (canceled)

7. A method for prevention and/or treatment of Porphyromonas gingivalis infection, comprising administering to a subject a pharmaceutically effective amount of killed Enterococcus faecalis.

8-9. (canceled)

10. A method for prevention and/or treatment of a disease attributable to Porphyromonas gingivalis, comprising administering to a subject a pharmaceutically effective amount of killed Enterococcus faecalis.

11-12. (canceled)

13. A method for inhibition of the growth of Porphyromonas gingivalis, comprising administering to a subject a pharmaceutically effective amount of killed Enterococcus faecalis.

14-15. (canceled)

16. A method for inhibition of gingipain activity, comprising administering to a subject a pharmaceutically effective amount of killed Enterococcus faecalis.

17-18. (canceled)
